(19) United States
(12) Patent Application Publication
(10) Pub. No.: US 2008/0262581 A1
(43) Pub. Date: Oct. 23, 2008

(54) CURRENT DENSITY DETECTION AND CONTROL SYSTEM AND METHOD FOR AN ELECTROKINETIC DELIVERY OF MEDICAMENTS

(75) Inventors: Michael S. Barsness, Oxford, MA (US); Edward J. Doherty, Mansfield, MA (US)

Correspondence Address: NIXON & VANDERHYE, PC, 901 NORTH GLEBE ROAD, 11TH FLOOR, ARLINGTON, VA, 22203, US

(73) Assignee: Transport Pharmaceuticals, Inc., Framingham, MA (US)

(21) Appl. No.: 12/104,515

(22) Filed: Apr. 17, 2008

Related U.S. Application Data
(60) Provisional application No. 60/912,261, filed on Apr. 17, 2007.

(51) Int. Cl. A61N 1/00

(52) U.S. Cl. 607/115

(57) ABSTRACT

An apparatus to monitor current density in the application of medicament to a treatment site of a mammalian user of a electrokinetic device including: an applicator cartridge including an active electrode, a matrix carrying a medicament or a medicament and an electrically conductive carrier; a device including an electrical power source connectable to the active electrode, a counter electrode, and an electronic circuit configured to control the application of electrical current through the active electrode to establish a conductive path extending from the power source, through the active electrode, matrix, the treatment site, the user and the counter electrode electrically connected to the power source, and an array of contacts monitoring current density flowing through the matrix and to the treatment site, wherein the array of sensors are arranged monitor the current density at various locations of the matrix and a contact area between the matrix and skin above the treatment site.

### RELATED APPLICATION

This application claims the benefit of the earlier filed U.S. Provisional Patent Application Ser. No. 60/912,261, filed Apr. 17, 2008.

### BACKGROUND OF INVENTION

The present invention relates generally to applicators for electrokinetic mass transfer of substances to live tissue and particularly relates to an apparatus for electrokinetically delivering substances, e.g., a medicament, to a treatment site under skin.

Electrokinetic delivery of medicaments apply medication locally through a human individual's skin to a treatment site. One type of electrokinetic delivery mechanism is iontophoresis, i.e., the application of an electric field to the skin to enhance the skin's permeability and to deliver various ionic agents, e.g., ions of salts or other drugs to the treatment site. Iontophoretic or transdermal or transmucosal cutaneous delivery techniques have obviated the need for hypodermic injection of many medicaments thereby eliminating the concomitant problem of trauma, pain and risk of infection to the individual. Other types of electrokinetic delivery mechanisms include electroosmosis, electroporation, and electromigration, any or all of which are more generally known as electrotransport, electromolecular transport or iontophoretic methods, all of which are collectively known as electrokinetic methods.

Electrokinetic devices have been developed for the private self administration of medicaments and for diagnostic application by an individual at non-medical or non-professional facilities. U.S. Pat. No. 6,792,306 and US Published Patent Application No. 2006/0167403, disclose electrokinetic delivery devices which include a housing containing a power source, electronics and a counter electrode, the device being shaped and configured for releasable securement to an individual's finger and terminating in an applicator head having an active electrode and medicament matrix. By applying the applicator head to the skin overlying the treatment site and with the medicament or a medicament and a carrier therefor carried by the applicator head, the medicament may be electrokinetically delivered to the treatment site.

The applicator head of the electrokinetic device is typically releasable. The head may include a cartridge containing a medicament matrix, e.g., pad, an active electrode(s) and connecting prongs that fit into a receiver of the device. The user inserts the prongs of a cartridge into the receiver of the device, removes a lid seal from the face of the cartridge to expose the medicament matrix, applies the matrix to the treatment site and activates the device to deliver the medicine through skin to the site. After the medicament is delivered to the treatment site, the user removes the cartridge from the device, and reinserts another cartridge for a subsequent application of medicament to another user, another treatment site, or to the same treatment site at a later application time.

The electrokinetic device typically applies a constant current to an active electrode(s) and medicament matrix in the applicator head. The current ionizes a medicament formulation in the matrix to leverage ionic forces to promote transfer of medication through the stratum corneum of the skin at the treatment site. The current from the matrix is assumed to flow uniformly from the front surface of the matrix, through a contact area between the matrix and skin and to the treatment site. To achieve uniform current, the impedance of the matrix and skin at the treatment site should be uniform. Uniform current provides a uniform current density in the matrix and at the skin contact area.

The delivery of medicament from the matrix to the treatment site is dependent on the current applied by the active electrode to the matrix. Current is a design factor in determining the delivery of medicament from the matrix to the skin and treatment site. The proper current may be determined based on the size of the applicator electrode, the matrix size and the required current per area needed to deliver the medicament. The proper current is also determined to avoid current burns, electrical shock and other damage or pain at the skin area to be in contact with the medicament matrix.

It is typically assumed that a uniform current distribution will be applied across the medicament matrix, skin contact area and treatment site. This assumption requires that irregularities in the impedance of the skin at the contact area be ignored. However, slight imperfections in the skin, such as due to skin diseases, lesions, and impaired skin tissue, may result in a localized reduction in the impedance at the contact area. Variations in the impedance of the skin at the treatment site disrupts the uniformity of current to the treatment site and can cause current to concentrate at small locations on the skin. Concentration of current, if severe, may result in patient burn, a pain and damage at the skin contact area.

There is a long felt need for systems and methods that detect concentration of current applied by a medicament matrix to a skin contact area, and a treatment site in an electrokinetic device. There is also a long felt need for systems and methods to provide a uniform flow of current through a medicament matrix, skin and to a treatment site to avoid unsafe concentration of currents at the skin area in contact with the matrix.

### SUMMARY OF INVENTION

Systems and methods have been developed for detecting uneven current densities in current flowing from a medicament matrix, through skin in contact with the matrix and to a treatment site. These systems and methods may also control the current to the medicament matrix to make more uniform the current densities applied to the matrix, skin contact area and treatment site.

The systems and methods may include zones in the active matrix and medicament matrix. The current to each of these zones may be individually controlled by separate active electrodes in each of the zones. The control may include adjusting the current to individual active electrodes such that the current to all zones of the medicament matrix is relatively uniform or disabling current to a zone associated with an excessive current density. The control may include increasing current at zones that have a relatively low current density and decreasing current having relatively high density to make the current density more uniform across the entirety of the skin contact area.

An apparatus is disclosed to monitor current density in the application of medicament to a treatment site of a mammalian user of a electrokinetic device, the apparatus comprising: an applicator cartridge including an active electrode, a matrix carrying a medicament or a medicament and an electrically conductive carrier; a device including an electrical power source connectable to the active electrode, a counter electrode, and an electronic circuit configured to control the application of electrical current through the active electrode to establish a conductive path extending from the power source, through the active electrode, matrix, the treatment site, the user and the counter electrode electrically connected to the power source, and an array of contacts monitoring current density flowing through the matrix and to the treatment site, wherein the array of sensors are arranged monitor the current density at various locations of the matrix and a contact area between the matrix and skin above the treatment site.

An apparatus is disclosed to monitor current density in the application of medicament to a treatment site of a mammalian user of a electrokinetic device, the apparatus comprising: an applicator cartridge including an active electrode, a matrix carrying a medicament or a medicament and an electrically conductive carrier; a device including an electrical power source connectable to the active electrode and a counter electrode, and an electronic circuit configured to control electrical current through the active electrode to establish a conductive path extending from the power source, through the active electrode, matrix, the treatment site, and the counter electrode electrically connected to the power source; an array of contacts monitoring current at various locations of the matrix, and a current control circuit adjusting the current through the matrix if the voltage potential on one contact of the array of contacts is substantially different than a voltage potential of at least one of the other contacts in the array of contacts.

A method is disclosed to monitor electrical current while applying medicament to a treatment site of a mammalian user of a electrokinetic device, the method comprising: placing over the treatment site and on skin of the user, a matrix carrying a medicament or a medicament with an electrically conductive carrier; applying electrical power to an active electrode electrically coupled to the matrix to establish a current flow from the active electrode, through the matrix and skin and to the treatment site; monitoring the current flow at a plurality of locations of the matrix; determining if the current flow through one location of the plurality of locations is substantially different that the current flow through another location of the plurality of locations, and reducing the current if there is a substantial difference in the current flow.

### DESCRIPTION OF THE DRAWINGS

The drawings included with this application and identified below illustrate embodiments of the disclosed invention and disclose the best mode of the invention now known to the inventors.

FIG. 1 is a perspective view of an exemplary electrokinetic delivery device including an applicator cartridge head secured to the delivery device.

FIG. 2 is a perspective view similar to FIG. 1 illustrating the device with one side of the housing removed to show internal components.

FIG. 3 is an exploded perspective view of the applicator cartridge head as viewed from its rear and side.

FIG. 4 is an exploded perspective view of the front and side of the applicator cartridge head illustrating a lid, matrix and cartridge head.

FIG. 5 is a side view of the applicator cartridge head.

FIG. 6 is an enlarged cross-sectional view of the applicator cartridge head secured to the device.

FIG. 7 is a schematic view of a medicament matrix pad, skin contact area, active electrode array, and a current density detection circuit coupled to the active electrode array.

FIG. 8 is a schematic view of an array of active electrodes, a medicament pad, a skin contact site, and a current density detection and current distribution circuit coupled to the active electrode array.

FIG. 9 is a schematic view of a medicament pad, skin contact site, array of active electrodes, and another embodiment of a current density detection and current distribution circuit coupled to the active electrode array.

FIG. 10 is a side view of a medicament matrix and shows schematically a uniform electrical current density flowing through the matrix from a plurality of active electrodes to a skin contact site.

FIG. 11 is a side view of a medicament matrix and shows schematically an asymmetrical electrical current density flowing through the matrix from a plurality of active electrodes to a skin contact site.

FIG. 12 is a front view of a medicament matrix, active electrode (which may be an array of active electrodes), and current contacts abutting a side periphery of the matrix. FIG. 12 also illustrates the effective resistances in the matrix between the active electrode and sensor contacts.

FIGS. 13 and 14 are front and side views, respectively, of a schematic illustration of a uniform current density distribution through a medicament matrix, where the skin contact area is relatively small as compared to the matrix.

FIGS. 15 and 16 are front and side views, respectively, of a schematic illustration of a uniform current density distribution through a medicament matrix wherein the skin contact area is relatively large as compared to the matrix.

FIGS. 17 and 18 are front and side views, respectively, of a schematic illustration of an asymmetrical current density distribution through a medicament matrix, where the skin contact area is offset from an axial center of the matrix.

FIGS. 19 and 20 are front and side views, respectively, of a schematic illustration of an asymmetrical current density distribution through a medicament matrix, where the current density is concentrated at a low resistance skin lesion in the contact area and the density is reduced at other regions of the skin contact area.

FIG. 21 is a electronic schematic diagram of an embodiment of an electronic circuit for monitoring current density in the medicament matrix of a electrokinetic device.

### DETAILED DESCRIPTION OF THE INVENTION

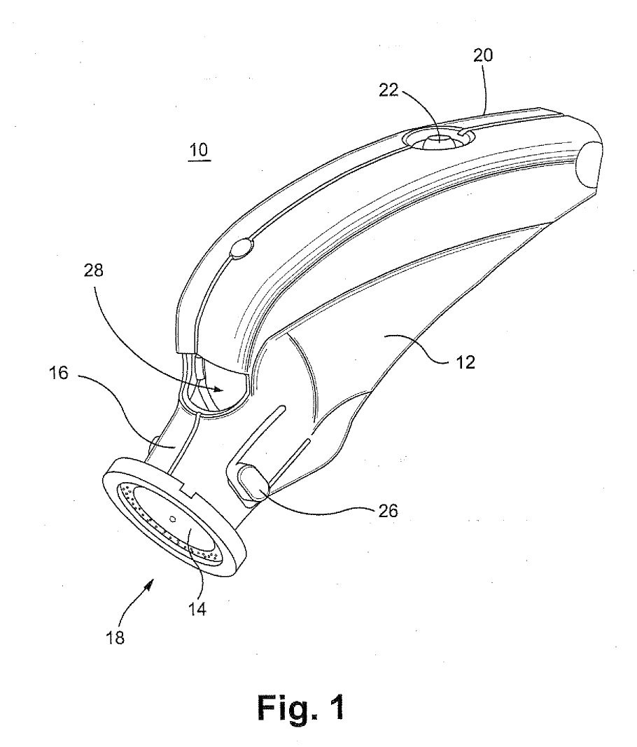

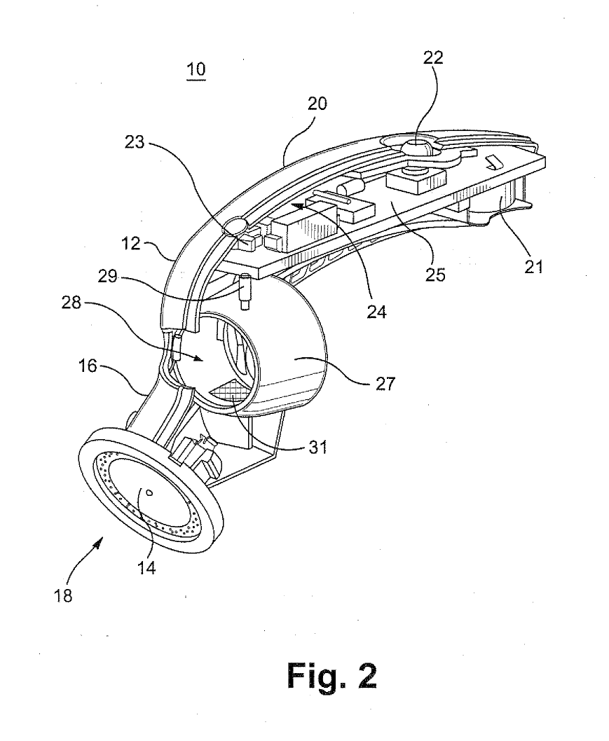

FIGS. 1 and 2 show a portable, self contained, lightweight, compact, finger mounted, electrokinetic medicament delivery device or medicator 10 (collectively a “device”) for application to a treatment site (TS) immediately below the skin of an individual. The device 10 includes a housing 12 mountable to an individual's finger with the distal end 16 of the device 10 mounting an applicator cartridge 18 containing an active electrode. The device 10 drives, e.g., electrokinetically transports, medicament interposed between the active electrode 14 and the individual's treatment site into the treatment site upon completion of an electrical circuit through the device, the active electrode, the medicament or hydration material carrying the medicament (collectively referred to as “medicament”), the individual's body and a counter electrode, i.e., tactile electrode carried by the device. While FIG. 1 shows the active electrode 14 exposed on the front face of the cartridge 18, the electrode is typically covered by a medicament matrix that is attached to a front face of the cartridge head and applied to the treatment site. The device 10 carrying the applicator cartridge 18 provides a facile and fatigue-free approach to the affected treatment site.

The housing 12 may include an internal compartment 24 for receiving a printed circuit board 25 containing a battery power source 21. The printed circuit board 25 or other electronic package may control current applied to the active electrode, time of current delivery to the active electrode, provide redundant safety features (such as a switch to prevent application of the current without a cartridge head in the device), and ensure user visual and/or audible signaling during use of the device, e.g., activation of the circuit board LED transmitted through the light pipe 23. A microprocessor on the printed circuit board may be programmed to control the application of current to the active electrode 14 and may receive signals, e.g., voltage levels from contacts or sensors monitoring the density and uniformity of current in the matrix and being applied to the skin contact area.

A proximal portion 20 of the housing 12 is elongated and shaped to fit comfortably on the top of the user's index finger. Located on the top surface of the housing is a manually actuated switch button 22 for energizing the circuitry and preparing the device for use. An opening 28 in the housing provides an access port through which long fingernails may extend. The opening allows finger nails to project through the housing so that good contact may be made between the fleshy pad of the finger and the ring 27. The opening facilitates proper contact with the counter electrode, e.g., contact surface 31, on an inside surface of the ring 27. Electrical connection between the contact surface 31 and the electronic circuits on the printed circuit board 25 is provided by a spring biased post 29 extending between the board 25 and an electrical contact 31 on the ring 27.

Adjacent to either side of the fingernail port 28 are ejector buttons 26 that, when depressed, disengage the applicator and provide a forward movement of the applicator cartridge away from the device during disposal. The exact size and position of the ejection and release features may be varied in response to the size of the applicator. The ejection and release features work in concert with the applicator and prevent inadvertent ejection or release during use.

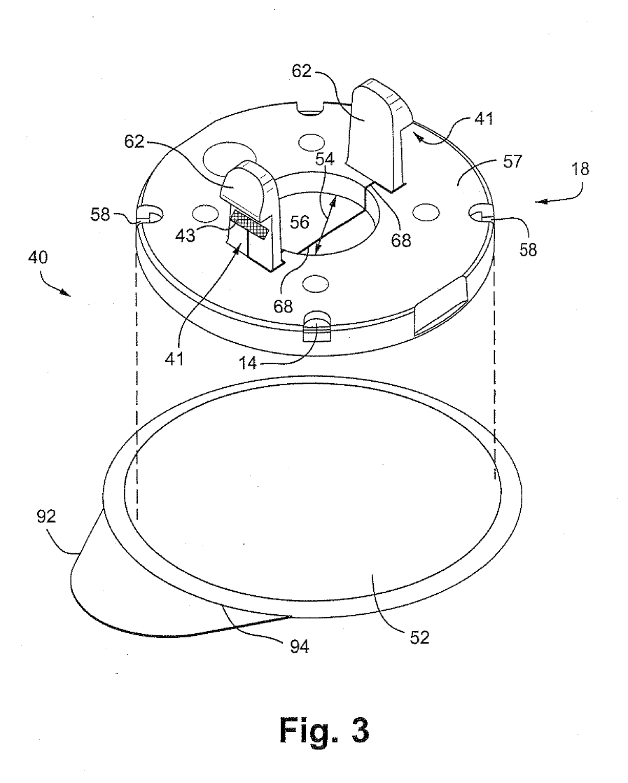

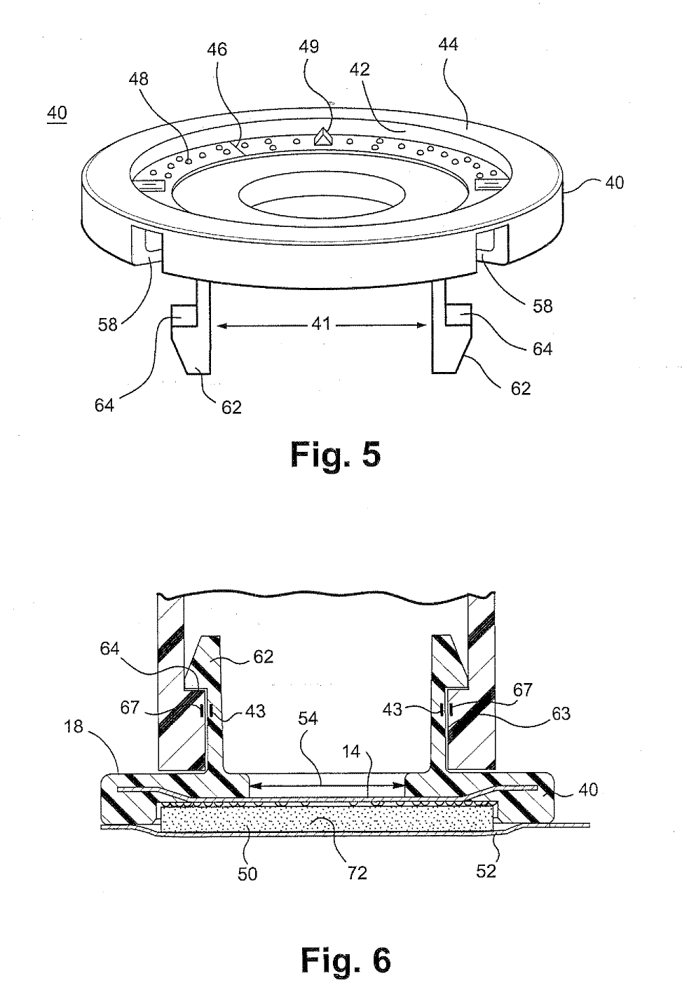

As shown in FIGS. 3 to 6, the applicator cartridge head 18 is preferably secured to the distal end 16 of the device 10 by a releasable coupling 41, e.g., prongs 62. By providing a two part device 10, e.g., housing 12 and cartridge 18, a fresh applicator cartridge 18 can be applied to the housing 12 for each subsequent use of the device 10. The used applicator cartridge 18 can be ejected or released from the housing by a one-hand operation. A new applicator cartridge head is attached to the device for subsequent treatments.

The applicator 18 generally includes an applicator cartridge head 40, e.g., a disc, having on its back or rear side a locking element 41 for securing the applicator 18 to a receiver in the distal end 16 of the device 10. The cartridge head 40 includes a circular recess 42 on a forward face of the head and defined by a rim 44 of the head 40. An active electrode surface 14 is disposed on a base 47 within the recess 42. The electrode surface may be a separate metal part, a metallized coating on one or more regions of the recess, or a conductive polymer attached to the applicator head. The active electrode may also be an array of electrodes arranged at various positions, e.g., zones, on the base 47.

The base 47 within the rim 44 of the applicator may be shaped to create a concave recess or other profile complementary to the shapes of the active electrode and matrix. The perimeter of the active electrode 14 may not extend to the inner wall of the rim 44. An annular surface 46 of the recess may remain between the rim and active electrode. A multitude of electrode sizes, shapes, and materials may be used to provide electrical contact between the applicator recess and the matrix. The annular surface 46 may include a plurality of raised projections, e.g., raised dimples 48, ridges 49 or combinations thereof of variable heights projecting from the surface 46. The dimples 48 may be radially spaced from each other across the surface 46.

The active electrode 14 may be composed of metal, a metallized polymer or a conductive polymer such as polyaniline, polypyrrole, or a polymer rendered conductive by means of a conductive dopant. The lid 52 over the front face of the head 40 may be composed of a polymer laminate with or without a metallic layer. The head 40 may be formed of a polymeric material, such as polypropylene or other polymer inert to the drug formulation in the medicament matrix 50.

To provide an electrical connection between the active electrode 14 of the applicator 18 and the power source, the opposite or second face, e.g., the backside 57, of the head 40, has an opening 54, preferably central to the head 40 through which the backside of the active electrode 14 is exposed. An electrical connection 68 is provided between the backside portion 56 of the electrode 14 and the circuit board when the applicator 18 is secured to the device. The backside 57 of applicator cartridge head 40 also includes one or more openings 58 which also expose a portion of the active electrode 14. The additional exposure of the electrode 14 facilitates the transmission of electrical signals for diagnostic testing during manufacture of the applicator 18.

The backside 57 of applicator cartridge head 40 includes a pair of prongs 62 that form one part of the locking element 41. The distal end 16 of device 10 mounts a pair of flats 64 along an inner surface of the distal end 16. The flats are a second part of the locking element 41. By inserting the prongs within the open inner surface of distal end 16, the prong heads engage the device flats to secure the applicator 18 to the device.

The active electrode 14 makes electrical contact with the circuit board within the receiver of the distal portion 16. The outer surface of the prongs 62 and an inner cylindrical wall 63 of the distal end 16 may be both electroplated to include a conductive pad 43 on outer surface of the prong and a conductive pad 67 on the inner wall 63 of the distal portion. A conductive line 68 on the surfaces of the head 40 provide an electrical path between the pad 43 on the prongs 62 and the back contact surface 56 of the active electrode. The conductive line 68 may be a bus that includes multiple conductive paths to various electrodes on an array of active electrodes.

When the prongs 62 are inserted in the distal portion 16, the prong and distal portion surfaces to abut provide an electrical contact through the contact pads 43, 67 between the circuit board 25 in the housing and the active electrode 14. The contact pads may be segmented to provide independent conductive paths for each active electrode in an array of active electrodes. Further, the surface(s) of the prongs may carry indicia or other markings for lot traceability, medicament identification, prevention of reuse of the applicator, or other information that is “read” by the microprocessor controlled circuitry in the device.

The applicator 18 also includes the matrix 50 which, is characterized by high void volume and does not interact with the medicament or the electrokinetic delivery of the medicament. The matrix 50 is a carrier supporting the medicament. Acceptable materials for the matrix include but are not limited to variable loft nonwoven and woven materials such as melt-blown, needlepunched, spunbonded, spunlaced or other processed natural fibers, polyolefin, polyester, rayon, nylon, and blends of these, reticulated polyether and polyester polyurethane foams, and silicone foams. Low void volume materials may also be used such as crosslinked hydrogels, interpenetrating polymer networks, scaffolds for immobilizing the active prior to iontophoretic release, highly viscosified formulations, and other matrices that do not rely upon a delivery from a liquid formulation. The matrix may also contain functional components 70 such as reinforcing scrims, networks, and other support structures to facilitate manufacture of the finished product. These layers may also be conductive to ensure homogeneous electrical contact with the drug formulation contained in the matrix. Additionally, the matrix may contain one or more layers carrying arrays of microneedles 72 or other surface features designed to physically penetrate the stratum corneum and promote delivery of medicaments intradermally or transdermally.

The porous matrix 50 may be a porous pad, membrane or substrate for the medicament. Acceptable materials for the porous matrix may include but are not limited to variable loft nonwoven and woven materials such as melt-blown, needlepunched, spunbonded, spunlaced or other processed polyolefin, polyester, rayon, nylon, and blends of these, reticulated polyether and polyester polyurethane foams, and silicone foams. Portions of the porous matrix may be conductive to ensure homogeneous electrical contact.

The matrix 50 is attached to the head 40 through mechanical or thermal bonding to a plurality of projections 48, 49. The head 40 may also be constructed with a porous surface to allow penetration and mechanical bonding of matrices such as hydrogels and other materials with low cohesive strength that are cast, injected, thermally formed, or otherwise inserted into the head during assembly. The matrix 50 may be porous to support the medicament and a hydration material that carries the medicament into the treatment site upon application of a current by the active electrode. The matrix 50 may be non-porous and carry a medicament that is driven directly by current into the treatment site. In the case of matrices containing thermoplastic fiber content, the matrix may be ultrasonically attached to the head 40 at one or more locations determined by contact with the array of raised projections, e.g., dimples 48 and bars 49.

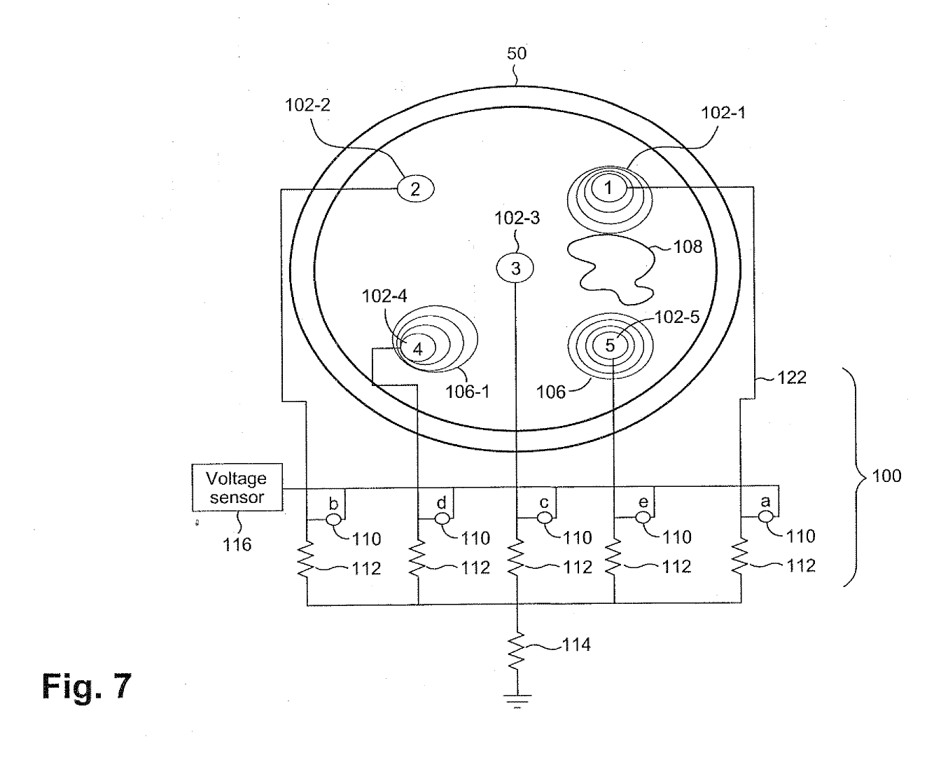

FIG. 7 is a schematic diagram of a sense circuit 100 monitoring current through the active electrodes 102-1 to 102-5 that form an array of active electrodes. Current is supplied to the active electrodes from a power source, flows through the medicament matrix 50 and the stratum corneum (outer layer of skin) and to the treatment site. The power supply circuits for applying current to the primary electrodes 102 are not shown in FIG. 7, but may include conductive elements in contact with the printed circuit board and power supply.

The current density at each active electrode is represented by current flow field lines 106 around each of the electrodes (for simplicity of illustration field lines are shown only around electrodes 102-1, 102-4 and 102-5). A uniform current density is represented by field lines of concentric circles 106 around an electrode, e.g., electrode 102-5. Non-uniform current densities are represented by field lines of circles offset with respect to the electrodes, e.g., electrodes 102-1 and 102-4. A non-uniform current density may occur due to variations in the skin, e.g., lesions 108, that result in a non-uniform impedance of the skin in contact with the matrix.

The sense circuit 100 includes, for each electrode 102, a contact element 110 (a to d) and a resistor 112. The sense circuit is grounded through a common resistor 114. The voltage potential at each contact element 110 is proportional to the current through the respective electrode 102. A voltage sensor 116 (see FIG. 21) included in the printed circuit board of the device monitors a voltage at the contact element 110, and indirectly monitors the current through the respective primary electrode 102.

The sensor circuit 100 is used to determine if the current is relatively uniformly distributed among the electrodes 102 of the active electrode. By monitoring the voltage levels at each contact element 110, the voltage sensor 116 and a microcontroller on the printed circuit board determine whether the current is evenly distributed among the active electrodes. The controller determines if one or more contact elements are at voltages above a threshold voltage, e.g., the voltage at one element 110 is at least twenty percent above an average voltage of all of the contact elements 110 or a comparison of voltages at two or more contact elements 110 shows substantial differences in voltages. If a contact element 110 is at a voltage above the threshold, the controller may disable the device and stop current being applied to the active matrix. Alternatively, the controller may reduce the current applied to all active electrodes to ensure that no one electrode operates at an excessive current level.

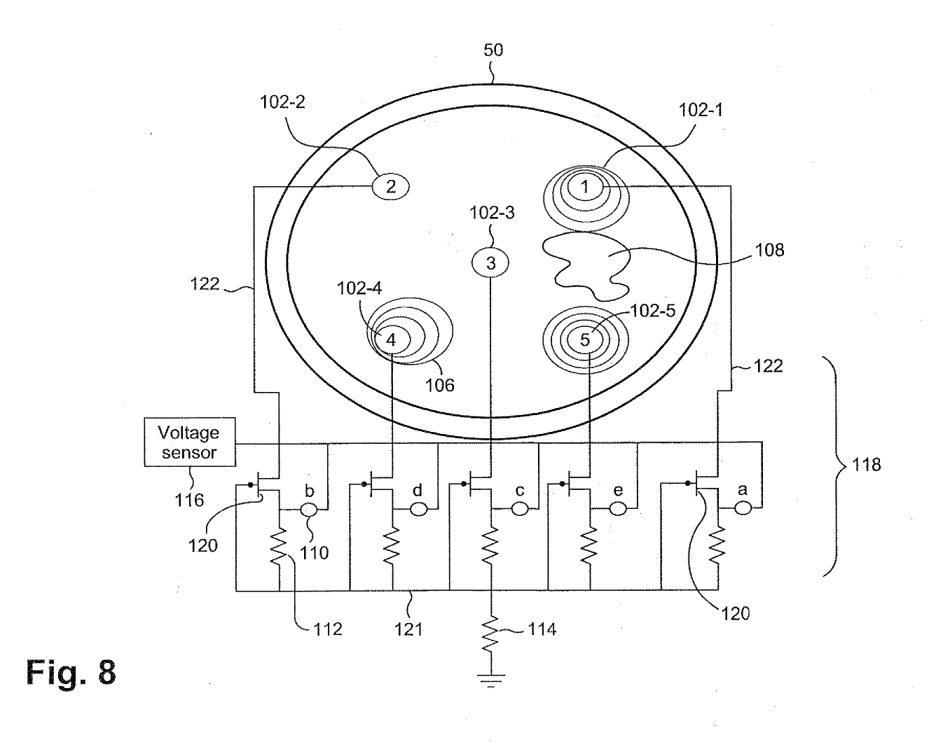

FIG. 8 is a schematic diagram of a sense and current adjusting circuit 118 which is similar to the sense circuit 100 except for field effect transistors (FETs) 120 that automatically adjust current in each of the active electrodes 102 of the active electrode array. The FETs are arranged such that the gains of the transistors 120 are each at a common voltage potential 121. The current through each of FET 120 will be substantially uniform with the other FETs due to the common voltage potential. Because the FETs ensure that each primary electrode 102 operates at a uniform current, variations of the impedance between the active electrodes 102 and the treatment site do not result in any substantial concentration of current at any one of the active electrodes. The FET (or other type of transistor) 120 ensures that a constant current flows through each of the circuit legs 122 connecting a respective primary electrode 102 to ground.

In addition, the voltage sensor 116 may monitor the voltages (and hence current) at each of the contact elements 110. If the voltage at any of the contact elements exceeds a threshold, the controller may disable the device by terminating current or reduce the current level applied to the active electrodes 102-1 to 102-5.

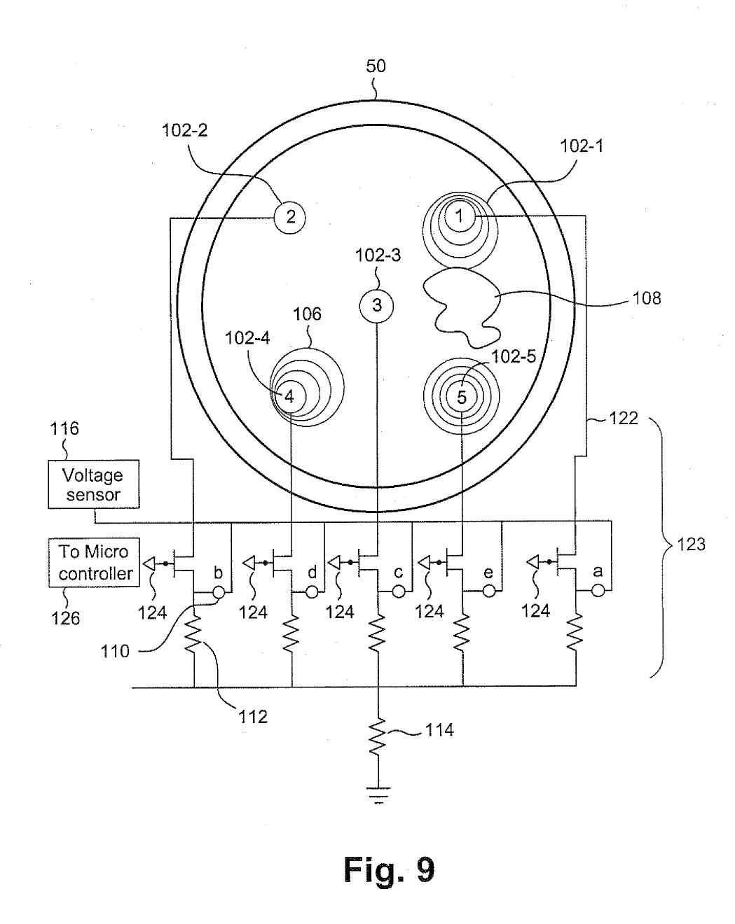

FIG. 9 is a schematic diagram showing a sense and current adjusting circuit 123 in which a series of FETs 120 that are switched 124 by a microcontroller 126. The gains of each of the FETs 120 are connected to the microcontroller which can switch 124 the voltage potential applied to the gains ON and OFF to control the FET, and hence the current through the respective active electrode 102. For example, the controller 126 may turn OFF an FET to prevent an excessive current density being applied to the skin by an active electrode 102-1 which has excessive current, as detected by the voltage sensor 116 monitoring the contact elements 110. If the sensor 116 detects a voltage above a threshold, the corresponding FET 120 may be turned off by the controller 126. The remaining FETs remain ON and current continues to flow to the remaining primary electrodes. Current will continue to flow to the matrix and skin areas corresponding to the turned OFF primary electrode, but at a reduced level due to more distant proximity to the current from the primary electrodes that remain active.

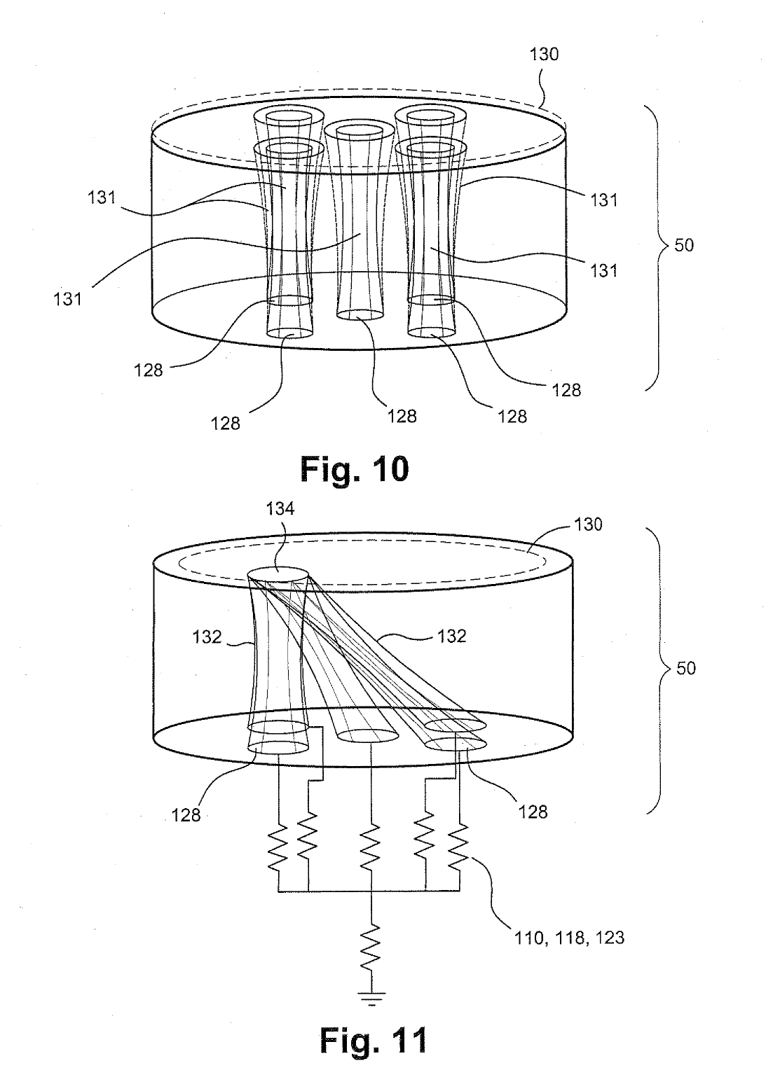

FIGS. 10 and 11 are schematic illustrations of a medicament matrix 50 having on a first side a plurality of active electrodes 128 that form an array of active electrodes arranged on a surface of the matrix. The side of the matrix that is placed in contact with the skin 130 is opposite to the side of the matrix in contact with the active electrodes 128. Current from the active electrodes flows through the matrix and skin 130 and to the treatment site (just below the skin). An even current distribution through the matrix 50 and skin is represented in FIG. 10 as an array of generally uniform current paths 131, where each path 131 extends from an active electrode 128 through the matrix 50 and skin 130 and to the treatment site. The current causes medicament in the matrix to flow through the skin and to the treatment site. An even distribution of current through the matrix (as shown in FIG. 10) provides a uniform application of medicament to the treatment site and avoids electrical burns on the skin. In contrast, the current paths 132 shown in FIG. 11 extend from the primary electrodes 128 to an area 134 on the skin having a low resistance, such as a skin lesion. The current through each of the active electrodes 128 is uneven because of variations in the current paths 132 from each of the primary electrodes to the lesion. Further, the current density becomes concentrated on the lesion and may result in pain or burns at the lesion. The sense circuit 110 and sense and current distribution circuits 118, 123, disclosed above, sense an uneven current distribution such as shown in FIG. 11 and, with respect to circuits 118, 123, may adjust the current applied to the active electrodes to cause the current distribution to be similar to the even distribution shown in FIG. 10.

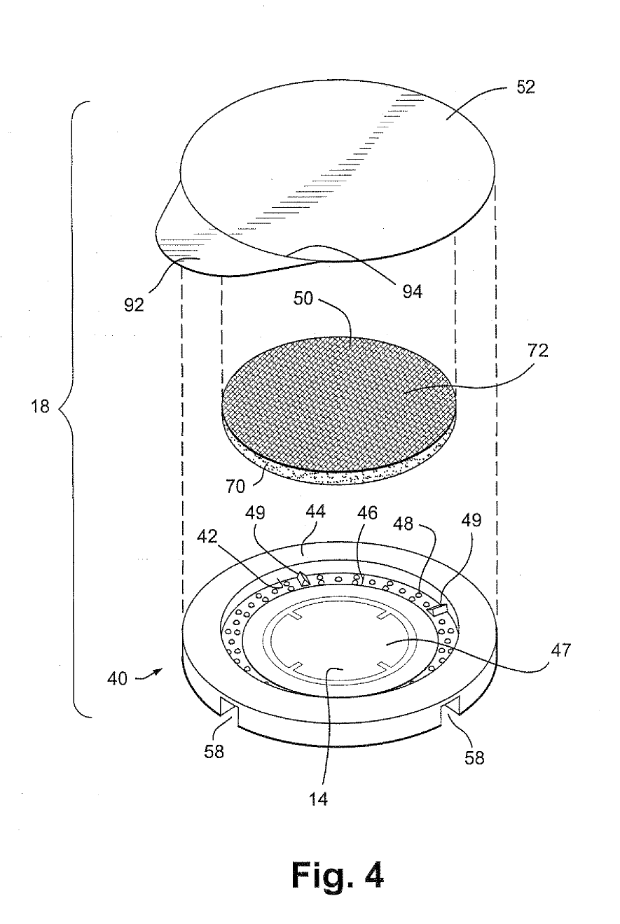

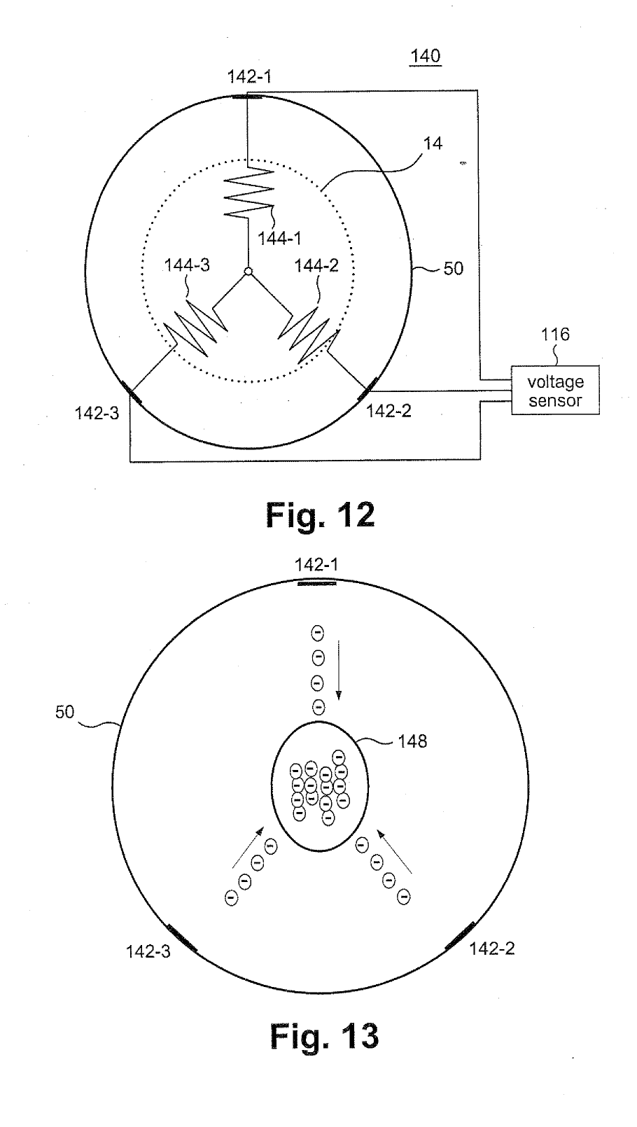

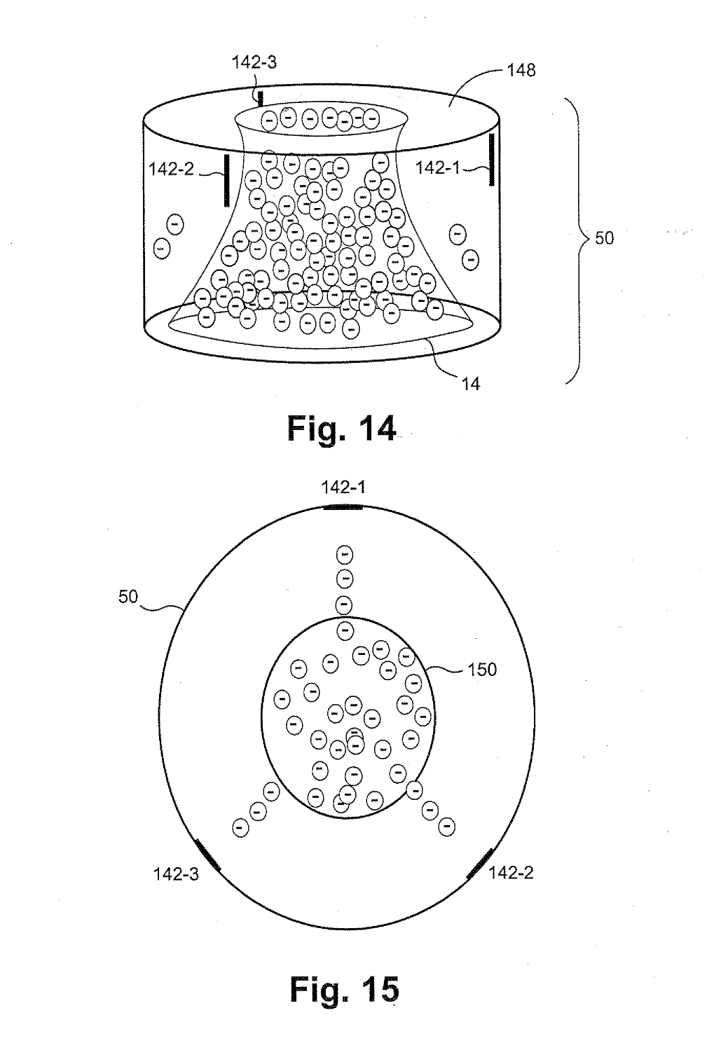

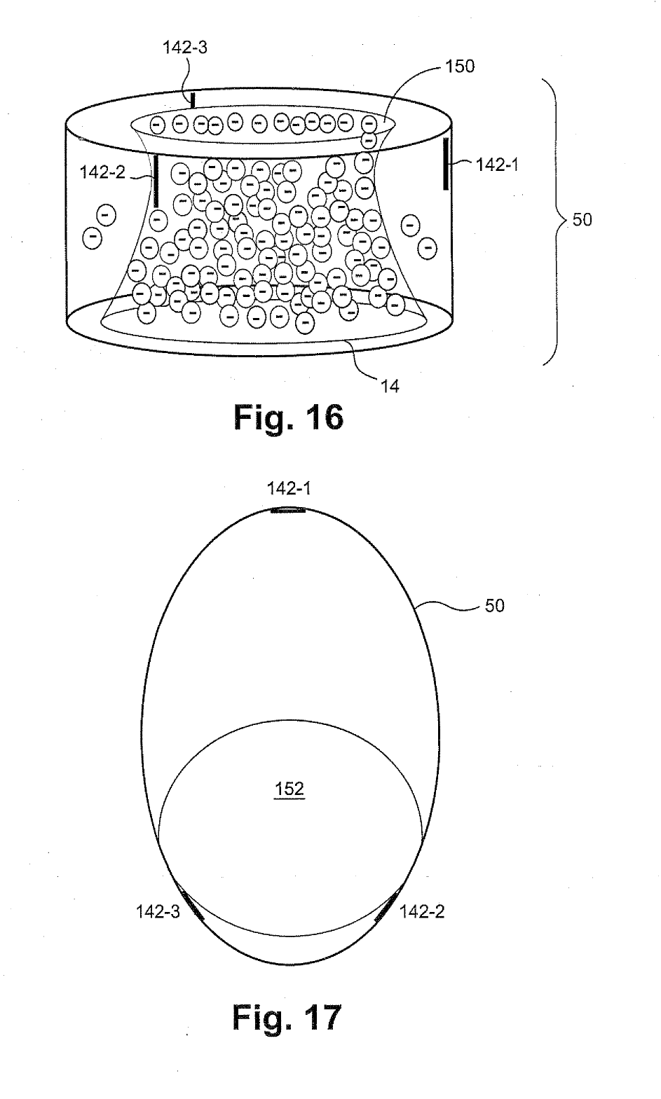

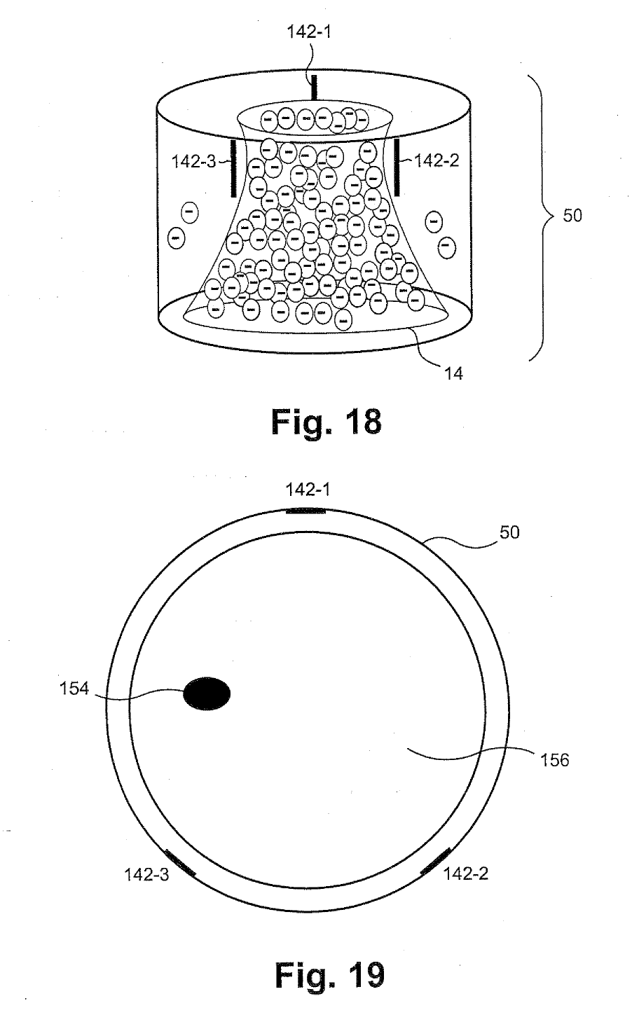

FIGS. 12 to 20 relate to a sensing circuit 140 that includes charge pads 142-1, 142-2 and 142-3, arranged on the cylindrical sidewall of a cartridge head supporting a medicament matrix 50. The charge pads 142 may be conductive contacts mounted on the inside wall of the rim 40 (see FIG. 4) and in abutting the side of the medicament matrix. The charge pads may be each connected to a voltage sensor 116 which monitors the voltage and indirectly the current at each of the charge pads 142.

FIG. 12 shows a front schematic view of the medicament matrix 50 and the charge pads 142. The active electrode 14 is shown by a dotted circular line and may be a metallic contact pad in the recess 42 (See FIG. 2) of the cartridge head 40 and abutting a surface of the matrix 50 opposite to the matrix surface applied to the skin.

The charge pads 142 are arranged at the periphery of the matrix and are each in contact with the matrix. An effective resistance 144, e.g., 144-1, 144-2 and 144-3, is show for illustrative purposes between the active electrode 14 and each of the charge pads 142. The resistance 144 represents the resistance of the matrix and particularly the resistance of the cream or gel (or other conductive medium) in the matrix. It is preferred that the resistance 144 through the conductive medium in the matrix and between the active electrode and each of the charge sensors is generally uniform.

The resistances 144-1, 144-2 and 144-3 between the active electrode 14 and each of the charge pads 142 are also dependent on the resistance of the skin in contact with the matrix. If a lesion or abrasion on the skin or other factor influences the resistance in the circuit between the active electrode and the treatment site, the voltage potential at one or more of the charge pads 142 will be similarly influenced.

FIGS. 13 to 16 show a uniform distribution of charged particles (represented by small circles with a minus sign) flowing from the active electrode 14 to a contact site 148, 150 on the skin of the user. If the skin contact area 148 is relatively small and generally centered on the matrix, a uniform current flow migrates (see arrows in FIG. 13) from the active electrode to the small contact area 148. The voltage potential at each of the charge pads 142 is low due to the small contact area 148. The potentials are uniform due to the contract surface 148 begin centered with respect to the matrix. If the skin contact area 150 is relatively large and centered on the matrix, a uniform current flow migrates (see arrows in FIG. 15) from the active electrode to the large skin contact area 150. If the current distribution through the matrix and to the treatment site is uniformly distributed through the surface of the skin in contact with the matrix, the voltage potential at each of the charge pad 142 should be the same. A large skin contact area 150 results in a higher voltage potential at each of the charge pad 142 as compared to the potentials associated with a small contact area 148.

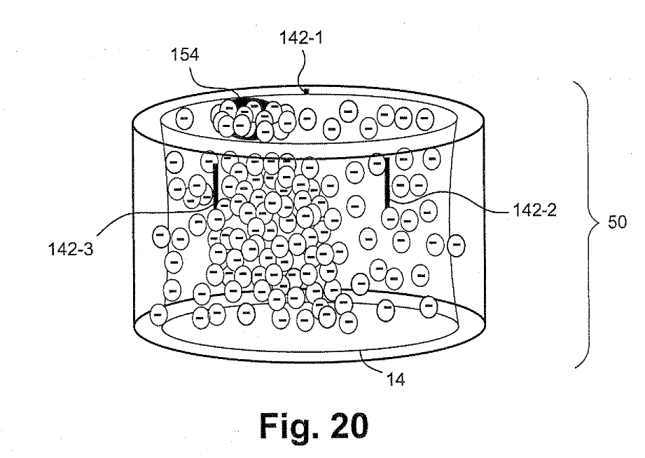

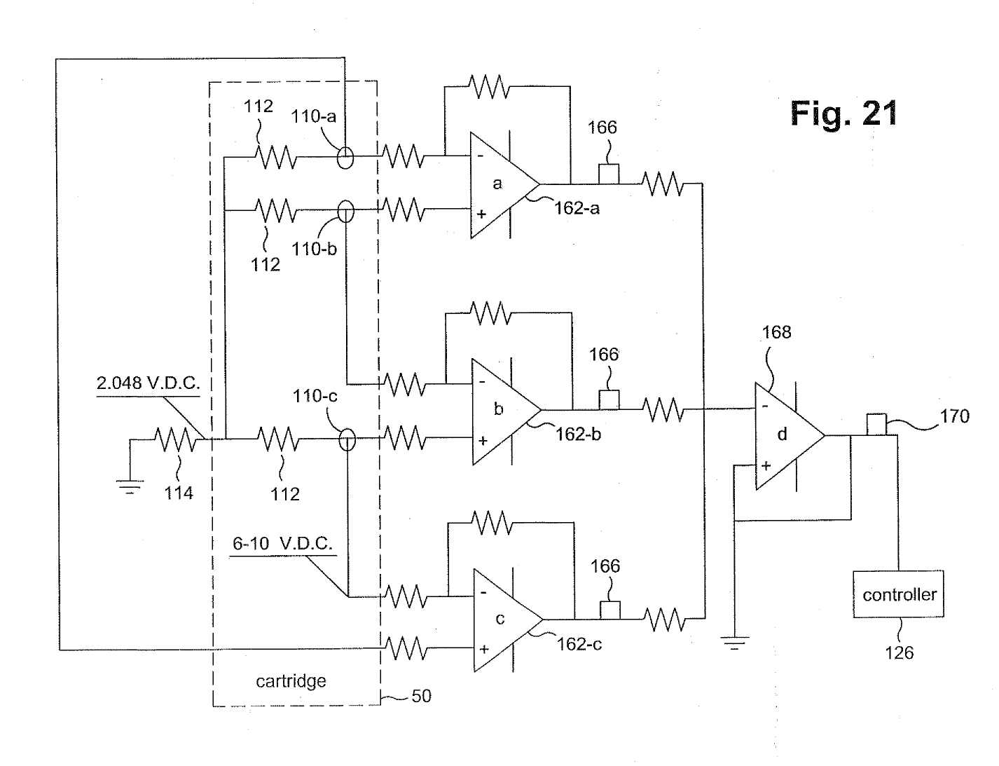

FIGS. 17 to 20 illustrate uneven charge distributions in the matrix 50 due to an offset contact area 152 between the skin and matrix. The offset contact area 152 causes electrical charges to be concentrated towards one side of the matrix and thus towards two of the charge pads 142-2, 142-3. The voltage potential of the other charge pad 142-1 will be lower than at the other charge pads. The uneven charge distribution, if excessive, may result in heating of the skin at the contact area 152, especially if the contact area is small. The uneven charge distribution may be due to a small contact area on the skin (see FIG. 17) or due to a lesion (See 154 in FIGS. 19 and 20). A lesion 154 or other artifact of the skin may have a low electrical resistance between the matrix and treatment site. Current tends to flow through the lesion due to its low resistance and increases the current density at the skin contact area near the lesion. This increased current density at the lesion could result in excessive heating at the lesion, if the device does not detect a high current density situation. As the current becomes concentrated near the lesion, the current density reduces through the matrix and the remaining portion of the skin contact area 156. If the current distribution is concentrated on one or point of the skin, and the voltage potentials at each of the charge pad 142 should differ due to the uneven charge distribution. For example, the voltage potential at pad 143-3 may be substantially higher than the potentials at pads 142-1 and 142-2.

A voltage sensor 116 monitors the voltage potential at each of the charge pad 142. The voltage output from each of the sensors may be compared by a controller 126 (see FIG. 9). If the voltage potentials measured by each charge pad 142 are generally the same, e.g., within 10% of the potential at another pad, the controller determines that the current density is generally uniform at the skin near the treatment site. If the voltage potentials as measured by one or more of the charge pad 142 are substantially different than the potentials reported by the other charge pads, the controller may determine that the current density at the skin may be concentrated and potentially could burn the skin. If a potential burn condition is detected, the controller may turn off current to the applicator cartridge or reduce the current to the application cartridge.

FIG. 21 is a schematic diagram of an exemplary electronic circuit 160 for the cartridge and printed circuit board 25 (FIG. 2). The electronic circuit includes the charge pads 110-a, 110-b, and 110-c (or 142). The circuit monitors the effective resistances between the treatment site on one side of the matrix 50 and the charge sensors. The voltage potentials at each of the charge pads are applied to operational amplifiers 162-a, 162-b and 162-c that each compare the voltage potential at two of the three charge pads. In particular, operational amplifier (a) compares the voltage potentials at charge pads 110-a and 110-b; operational amplifier (b) compares the voltage potentials at charge pads 110-b and 110-c, and operational amplifier (c) compares the voltage potentials at charge pads 110-c and 110-a. If the charge density through the matrix is uniformly distributed the differential outputs 166 from each of the operational amplifiers 162 should be at or near zero volts. If the charge pad, e.g., 110-a, is at a voltage potential substantially different than another pad, e.g., 110-b, the operational amplifier 162a monitoring both pads will output a substantially high output level. The output 166 from one or more of the operational amplifiers at a non-zero voltage level causes another operational amplifier 168 that amplifies the non-zero voltage level and outputs 170 the non-zero voltage level to the controller 126 on the printed circuit board. The controller 126 determines whether the output 170 is sufficiently large to disable the device to stop current to the matrix from the active electrode or to reduce the current level until the voltage output 170 is reduced to a predetermined acceptable level.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. An apparatus to monitor current density in the application of medicament to a treatment site of a mammalian user of a electrokinetic device, the apparatus comprising:
an applicator cartridge including an active electrode, a matrix carrying a medicament or a medicament and an electrically conductive carrier;
a device including an electrical power source connectable to the active electrode, a counter electrode, and an electronic circuit configured to control the application of electrical current through the active electrode to establish a conductive path extending from the power source, through the active electrode, matrix, the treatment site, the user and the counter electrode electrically connected to the power source, and
an array of contacts monitoring current density flowing through the matrix and to the treatment site, wherein the array of sensors are arranged monitor the current density at various locations of the matrix and a contact area between the matrix and skin above the treatment site.

2. An apparatus as in claim 1 wherein the array of contacts are an array of conductive pads arranged on a periphery of the matrix.

3. An apparatus as in claim 2 wherein the periphery of the matrix is cylindrical sidewall of the matrix and the array of contacts is mounted on the cartridge to abut the cylindrical sidewall of the matrix.

4. An apparatus as in claim 1 wherein the active electrode is an array of active electrodes spatially arranged to contact various locations of the surface and the array of contacts includes electrical contacts for each of the active electrodes in the array.

5. An apparatus as in claim 4 wherein the array of active electrodes is arranged on an inside surface of the cartridge and the inside surface of the cartridge abuts a surface of the matrix opposite to a surface of the matrix which contacts skin over the treatment site.

6. An apparatus as in claim 1 wherein the further comprising an electronic circuit coupled to the array of contacts, wherein the electronic circuit compares a voltage potentials at least two of the contacts in the array and generates an alarm if a difference of the voltage potentials of the at least two contacts exceeds a predetermined threshold limit.

7. An apparatus as in claim 1 wherein the further comprising an electronic circuit coupled to the array of contacts, wherein the electronic circuit compares a voltage potential between at least two of the contacts in the array and the circuit reduces current applied to the active electrode if a difference of the voltage potentials of the at least two contacts exceeds a predetermined threshold limit.

8. An apparatus as in claim 7 wherein the reduction in the current is a reduction to zero current.

9. An apparatus to monitor current density in the application of medicament to a treatment site of a mammalian user of a electrokinetic device, the apparatus comprising:
an applicator cartridge including an active electrode, a matrix carrying a medicament or a medicament and an electrically conductive carrier;
a device including an electrical power source connectable to the active electrode and a counter electrode, and an electronic circuit configured to control electrical current through the active electrode to establish a conductive path extending from the power source, through the active electrode, matrix, the treatment site, and the counter electrode electrically connected to the power source;
an array of contacts monitoring current at various locations of the matrix, and
a current control circuit adjusting the current through the matrix if the voltage potential on one contact of the array of contacts is substantially different than a voltage potential of at least one of the other contacts in the array of contacts.

10. An apparatus as in claim 9 wherein the current control circuit includes at least one operational amplifier comparing the voltage potentials of at least two of the contacts in the array of contacts.

11. An apparatus as in claim 9 wherein the current control circuit includes at least two operational amplifiers each comparing the voltage potentials of at least two of the contacts in the array of contacts, and another operational amplifier monitoring outputs form the at least two operational amplifiers.

12. An apparatus as in claim 9 wherein the current control circuit includes a field effect transistor in series with each of the contacts in the array of contacts, and the field effect transistors each having a gain connected to a common voltage potential.

13. An apparatus as in claim 9 wherein each of the contacts in the array of contacts has a conductive connection to a respective active electrode in an array of active electrodes.

14. A method to monitor electrical current while applying medicament to a treatment site of a mammalian user of a electrokinetic device, the method comprising:
placing over the treatment site and on skin of the user, a matrix carrying a medicament or a medicament with an electrically conductive carrier;
applying electrical power to an active electrode electrically coupled to the matrix to establish a current flow from the active electrode, through the matrix and skin and to the treatment site;
monitoring the current flow at a plurality of locations of the matrix;
determining if the current flow through one location of the plurality of locations is substantially different that the current flow through another location of the plurality of locations, and
reducing the current if there is a substantial difference in the current flow.

15. A method as in claim 14 wherein the current flow is monitored at a plurality of contact pads arranged around a periphery of the matrix.

16. A method as in claim 14 wherein the current flow is monitored at a plurality of contacts each coupled to one electrode in an array of the active electrodes.

17. A method as in claim 14 wherein the current flow is monitored by determining a voltage potential at each of the locations on the matrix.

18. A method as in claim 14 wherein reducing the current includes shutting off the current.

19. A method as in claim 14 wherein the active electrode is an array of active electrodes and reducing the current includes reducing current to one of the active electrodes in the array below the current applied to other active electrodes in the array.

20. A method as in claim 14 wherein reducing the current is performed by field effect transistors (FETs) each having gains at a common voltage potential.
